(19) United States
(12) Patent Application Publication
(10) Pub. No.: US 2019/0254732 A1
(43) Pub. Date: Aug. 22, 2019

(54) VACUUM PUMP ACTIVATION ASSEMBLY FOR CRYOGENIC BALLOON CATHETER SYSTEM

(71) Applicant: Cryterion Medical, Inc., Carlsbad, CA (US)

(72) Inventor: Chadi Harmouche, Saint-Laurent (CA)

(21) Appl. No.: 16/264,042

(22) Filed: Jan. 31, 2019

Related U.S. Application Data
(60) Provisional application No. 62/625,951, filed on Feb. 2, 2018.

(51) Int. Cl. A61B 18/02

(52) U.S. Cl. CPC A61B 18/02; A61B 2018/0022

(57) ABSTRACT

A catheter system comprises a balloon catheter configured to receive a cryogenic fluid and including a handle assembly having a handle connector, a control console including a console connector; a vacuum pump, and a vacuum pump activation system comprising a coupler including a first coupler connector and a second coupler connector, the first coupler connector configured to connect to the handle connector, the second coupler connector configured to connect to the console connector. A first connection sensor senses when the first coupler connector and the handle connector are connected, or when the second coupler connector and the console connector are connected. The first connection sensor generates a first sensor output. A control valve is positioned on the fluid exhaust line, and a controller receives the first sensor output, the controller selectively opening the control valve to activate the vacuum pump based upon the first sensor output.

### CROSS-REFERENCE TO RELATED APPLICATION

This application claims priority to Provisional Application No. 62/625,951, filed Feb. 2, 2018, which is herein incorporated by reference in its entirety.

### TECHNICAL FIELD

The present disclosure relates to medical devices and methods for treating cardiac arrhythmias. More specifically, the disclosure relates to devices and methods for cardiac cryoablation.

### BACKGROUND

Cardiac arrhythmias involve an abnormality in the electrical conduction of the heart and are a leading cause of stroke, heart disease, and sudden cardiac death. Treatment options for patients with arrhythmias include medications, implantable devices, and catheter ablation of cardiac tissue.

Catheter ablation involves delivering ablative energy to tissue inside the heart to block aberrant electrical activity from depolarizing heart muscle cells out of synchrony with the heart's normal conduction pattern. The procedure is performed by positioning the tip of an energy delivery catheter adjacent to diseased or targeted tissue in the heart. The energy delivery component of the system is typically at or near the most distal (farthest from the operator) portion of the catheter, and often at the tip of the device. Various forms of energy are used to ablate diseased heart tissue, including radio frequency, balloon cryotherapy which uses cryoballoons (also sometimes referred to herein as “balloon catheters”), ultrasound and laser energy, to name a few. In the case of balloon cryotherapy, a tip of the balloon catheter is positioned adjacent to target tissue, at which time energy is delivered to create tissue necrosis, rendering the ablated tissue incapable of conducting electrical signals.

Atrial fibrillation is one of the most common arrhythmias treated using cardiac ablation. Atrial fibrillation is typically treated by pulmonary vein isolation, a procedure that removes unusual electrical conductivity in the pulmonary vein. In the earliest stages of the disease, paroxysmal atrial fibrillation, the treatment strategy involves isolating the pulmonary vein(s) from the left atrial chamber. Recently, the use of balloon cryotherapy procedures and other types of ablation procedures to treat atrial fibrillation has increased. In part, this stems from ease of use, shorter procedure times and improved patient outcomes.

During cryoablation procedures, the catheter and the control console must be mechanically connected to allow the flow of cryogenic fluid from the control console to the catheter and back to the control console. Generally, the cryogenic fluid flows in a liquid phase from the catheter to the cryoballoon. The cryogenic fluid then undergoes a phase change and returns to the control console as exhaust in a gaseous phase with the assistance of a vacuum pump. During the cryoablation procedure, one use of the vacuum pump is to assist in detecting breaches in the area between an inner cryoballoon and an outer cryoballoon of the balloon catheter. Further, upon completion of the cryoablation procedure, the vacuum pump may be activated to assist in retracting the catheter back into a catheter sheath. For example, the vacuum pump may be used to remove cryogenic fluid from the catheter and/or deflate the inner and/or outer cryoballoons. Currently, after an operator connects the catheter to the control console, the operator needs to manually activate the vacuum pump, such as by pressing a button on the control console and/or a graphical display. This additional step to activate the vacuum pump once the control console and catheter are connected can increase the likelihood of operator error and/or cause an increase in the time of the cryoablation procedure.

### SUMMARY

Example 1 is a catheter system including a balloon catheter and a control console. The balloon catheter is configured to receive a cryogenic fluid and includes a handle assembly that includes a handle connector. The control console includes a console connector, a vacuum pump, and a vacuum pump activation system. The vacuum pump activation system includes a coupler, a first connection sensor, a control valve, and a controller. The coupler includes a first coupler connector and a second coupler connector, the first coupler connector being configured to connect to the handle connector, the second coupler connector being configured to connect to the console connector. The first connection sensor senses when at least one of (i) the first coupler connector and the handle connector are connected, and (ii) the second coupler connector and the console connector are connected. The first connection sensor generates a first sensor output. The control valve is positioned on the fluid exhaust line. The controller receives the first sensor output. The controller selectively opening the control valve to activate the vacuum pump based at least partially upon the first sensor output.

Example 2 is the catheter system of Example 1, wherein the first connection sensor is positioned within the handle connector.

Example 3 is the catheter system of Example 1, wherein the first connection sensor is positioned within the first coupler connector.

Example 4 is the catheter system of Example 1, wherein the first connection sensor is positioned within the console connector.

Example 5 is the catheter system of Example 1, wherein the first connection sensor is positioned within the second coupler connector.

Example 6 is the catheter system of any of Examples 1-5, wherein the control valve is located within the control console.

Example 7 is the catheter system of any of Examples 1-6, further comprising a second connection sensor that senses when at least one of (i) the first coupler connector and the handle connector are connected, and (ii) the second coupler connector and the console connector are connected, the second connection sensor generating a second sensor output.

Example 8 is the catheter system of Example 7, wherein the controller receives the second sensor output, the controller selectively opening the control valve to activate the vacuum pump based at least partially upon the second sensor output.

Example 9 is a vacuum pump activation system for a catheter system, the catheter system including (i) a handle assembly that includes a handle connector, (ii) a balloon catheter, the balloon catheter being configured to receive a cryogenic fluid, (iii) a control console that includes a console connector, and (iv) a vacuum pump. The vacuum pump activation system includes a coupler, a first connection sensor, a second connection sensor, a control valve, and a controller. The coupler includes a first coupler connector and a second coupler connector, the first coupler connector being configured to connect to the handle connector, the second coupler connector being configured to connect to the console connector. The first connection sensor senses when the first coupler connector and the handle connector are connected, the first connection sensor generating a first sensor output. The second connection sensor senses when the second coupler connector and the console connector are connected, the second connection sensor generating a second sensor output. The control valve is positioned on the fluid exhaust line. The controller receives the first sensor output and second sensor output, the controller selectively opening the control valve to activate the vacuum pump based at least partially upon the first sensor output and the second sensor output.

Example 10 is the vacuum pump activation system of Example 9, wherein the first connection sensor is positioned within the handle connector.

Example 11 is the vacuum pump activation system of Example 9, wherein the first connection sensor is positioned within the first coupler connector.

Example 12 is the vacuum pump activation system of any of Examples 9-11, wherein the second connection sensor is positioned within the console connector.

Example 13 is the vacuum pump activation system of any of Examples 9-11, wherein the second connection sensor is positioned within the second coupler connector.

Example 14 is the vacuum pump activation system of any of Examples 9-13 wherein the control valve is located within the control console.

Example 15 is a method for activating a vacuum pump. The method includes connecting a first coupler connector to a handle connector and a second coupler connector to a console connector, generating first sensor output with a first connection sensor, sending the first sensor output generated by the first connection sensor to a controller, and opening with the controller a control valve based at least partially upon the first sensor output.

Example 16 is the method of Example 15, wherein sending the first sensor output includes the first connection sensor being positioned within the handle connector.

Example 17 is the method of Example 15, wherein sending the first sensor output includes the first connection sensor being positioned within the first coupler connector.

Example 18 is the method of Example 15, wherein sending the first sensor output includes the first connection sensor being positioned within the console connector.

Example 19 is the method of Example 15, wherein sending the first sensor output includes the first connection sensor being positioned within the second coupler connector.

Example 20 is the method of any of Examples 15-19, further including generating second sensor output with a second connection sensor, and opening a control valve based at least partially upon the second sensor output.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a simplified schematic side view of a patient and an embodiment of a cryogenic balloon catheter system having features of the present disclosure;

FIG. 2 is a simplified schematic side view of another embodiment of the cryogenic balloon catheter system including one embodiment of a vacuum pump activation assembly;

FIG. 3 is a simplified schematic side view of still another embodiment of the cryogenic balloon catheter system including another embodiment of the vacuum activation assembly;

FIG. 4 is a simplified side view of yet another embodiment of the cryogenic balloon catheter system including still another embodiment of the vacuum pump activation assembly; and

FIG. 5 is a simplified side view of even another embodiment of the cryogenic balloon catheter system including yet another embodiment of the vacuum pump activation assembly.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

Embodiments of the present disclosure are described herein in the context of a vacuum pump activation assembly. Those of ordinary skill in the art will realize that the following detailed description of the present disclosure is illustrative only and is not intended to be in any way limiting. Other embodiments of the present disclosure will readily suggest themselves to such skilled persons having the benefit of this disclosure. Reference will now be made in detail to implementations of the present disclosure as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

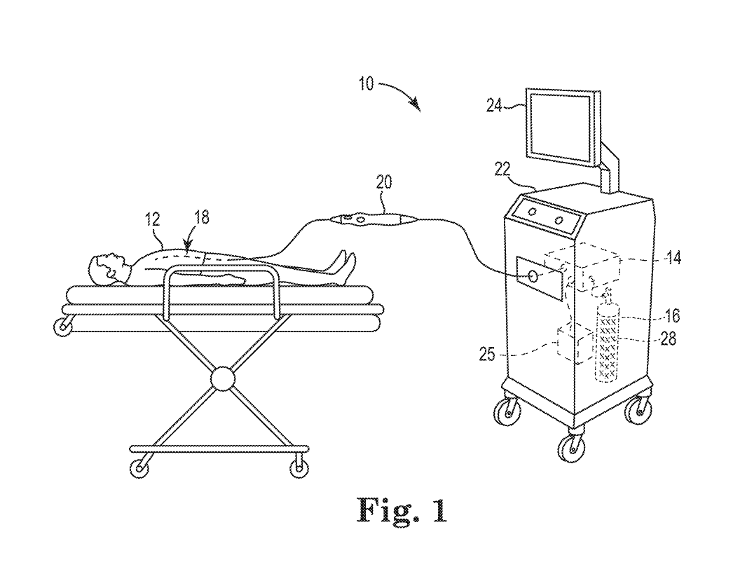

FIG. 1 is a schematic side view of one embodiment of a cryogenic balloon catheter system 10 (also sometimes referred to herein as a “catheter system”) for use with a patient 12, which can be a human being or an animal. The design of the catheter system 10 can be varied. Although the catheter system 10 is specifically described herein with respect to a cryogenic balloon catheter system, it is understood and appreciated that other types of catheter systems and/or ablation systems can equally benefit by the teachings provided herein. In certain embodiments, such as the embodiment illustrated in FIG. 1, the catheter system 10 can include one or more of a control system 14, a fluid source 16, a balloon catheter 18, a handle assembly 20, a control console 22, a graphical display 24 and a vacuum pump 25. It is understood that although FIG. 1 illustrates the structures of the catheter system 10 in a particular position, sequence and/or order, these structures can be located in any suitably different position, sequence and/or order than that illustrated in FIG. 1. It is also understood that the catheter system 10 can include fewer or additional components than those specifically illustrated and described herein.

In various embodiments, the control system 14 can control release and/or retrieval of a cryogenic fluid 26 to and/or from the balloon catheter 18. In certain embodiments, the control system 14 can control various structures described herein that are responsible for maintaining and/or adjusting a flow rate and/or a fluid pressure of the cryogenic fluid 26 that is released to the balloon catheter 18 during a cryoablation procedure. In various embodiments, the control system 14 can control activation and/or deactivation of one or more other processes of the balloon catheter 18 described herein. Additionally, or in the alternative, the control system 14 can receive data and/or other information (hereinafter sometimes referred to as “sensor output”) from various structures within the catheter system 10. In some embodiments, the control system 14 can assimilate and/or integrate the sensor output, and/or any other data or information received from any structure within the catheter system 10. Additionally, or in the alternative, the control system 14 can control positioning of portions of the balloon catheter 18 within the body of the patient 12, and/or can control any other suitable functions of the balloon catheter 18.

The fluid source 16 contains the cryogenic fluid 26, which is delivered to the balloon catheter 18 with or without input from the control system 14 during the cryoablation procedure. The type of cryogenic fluid 26 that is used during the cryoablation procedure can vary. In one non-exclusive embodiment, the cryogenic fluid 26 can include liquid nitrous oxide. In another non-exclusive embodiment, the cryogenic fluid 26 can include liquid nitrogen. However, any other suitable cryogenic fluid 26 can be used.

The balloon catheter 18 is inserted into the body of the patient 12. While specific reference is made herein to the balloon catheter 18, it is understood that any suitable type of catheter and/or medical device may be used. In one embodiment, the balloon catheter 18 can be positioned within the body of the patient 12 using the control system 14. Alternatively, the balloon catheter 18 can be manually positioned within the body of the patient 12 by a health care professional (also sometimes referred to herein as an “operator”). As used herein, health care professional and/or operator can include a physician, a physician's assistant, a nurse and/or any other suitable person and/or individual. In certain embodiments, the balloon catheter 18 is positioned within the body of the patient 12 utilizing the sensor output from the balloon catheter 18. In various embodiments, the sensor output is received by the control system 14, which then can provide the operator with information regarding the positioning of the balloon catheter 18. Based at least partially on the sensor output feedback received by the control system 14, the operator can adjust the positioning of the balloon catheter 18 within the body of the patient 12.

The handle assembly 20 is handled and used by the operator or other user to operate, position and/or control the balloon catheter 18. The design and specific features of the handle assembly 20 can vary to suit the design requirements of the catheter system 10. In the embodiment illustrated in FIG. 1, the handle assembly 20 is separate from, but in electrical and/or fluid communication with the control system 14, the fluid source 16 and/or the graphical display 24. In some embodiments, the handle assembly 20 can integrate and/or include at least a portion of the control system 14 within an interior of the handle assembly 20. It is understood that the handle assembly 20 can include additional components than those specifically illustrated and described herein.

In the embodiment illustrated in FIG. 1, the control console 22 includes the control system 14, the fluid source 16, the graphical display 24 and the vacuum pump 25. However, in alternative embodiments, the control console 22 can contain additional structures not shown or described herein. Still alternatively, the control console 22 may not include various structures that are illustrated within the control console 22 in FIG. 1. For example, in one embodiment, the control console 22 does not include the graphical display 24. In another embodiment, the control console 22 does not include the vacuum pump 25.

The graphical display 24 provides the operator or other user of the catheter system 10 with information that can be used before, during and after the cryoablation procedure. The specifics of the graphical display 24 can vary depending upon the design requirements of the catheter system 10, or the specific needs, specifications and/or desires of the operator or other user.

In one embodiment, the graphical display 24 can provide static visual data and/or information to the operator or other user. In addition, or in the alternative, the graphical display 24 can provide dynamic visual data and/or information to the operator or other user, such as video data or any other data that changes over time. Further, in various embodiments, the graphical display 24 can include one or more colors, different sizes, varying brightness, etc., that may act as alerts to the operator or other user. Additionally, or in the alternative, the graphical display can provide audio data or information to the operator or other user.

The vacuum pump 25 can remove, evacuate and/or retrieve some or all of the cryogenic fluid 26 from within the balloon catheter 18. The design and specific features of the vacuum pump 25 can vary to suit the design requirements of the catheter system 10. In the embodiment illustrated in FIG. 1, the vacuum pump 25 is separate from, but in electrical and/or fluid communication with the control system 14, the balloon catheter 18 and/or the handle assembly 20. In various embodiments, the vacuum pump 25 is always on. However, as used herein, when the vacuum pump 25 is activated, the vacuum pump 25 is pulling a vacuum and actively venting, removing, evacuating and/or retrieving some or substantially all of the cryogenic fluid 26 from within the balloon catheter 18. During cryoablation procedures, the vacuum pump 25 can be activated to remove, evacuate and/or retrieve some and/or substantially all of the cryogenic fluid 26 from the balloon catheter 18, for example, to maintain an appropriate balloon pressure within the balloon catheter 18 and/or to allow the balloon catheter 18 to be more safely removed from the patient 12. In contrast, in certain embodiments, the vacuum pump 25 may not be activated even though the vacuum pump 25 is on. As used herein, the vacuum pump 25 is not activated when it is not actively venting, removing, evacuating and/or retrieving some or substantially all of the cryogenic fluid 26 from the balloon catheter 18 during cryoablation procedures.

In the embodiment illustrated in FIG. 1, the vacuum pump 25 is integrated and/or included as part of the control console 22. In another embodiment, the vacuum pump 25 may be separate from the control console 22. Alternatively, in other embodiments, the vacuum pump 25 may be integrated and/or included as part of any other suitable structure within the catheter system 10.

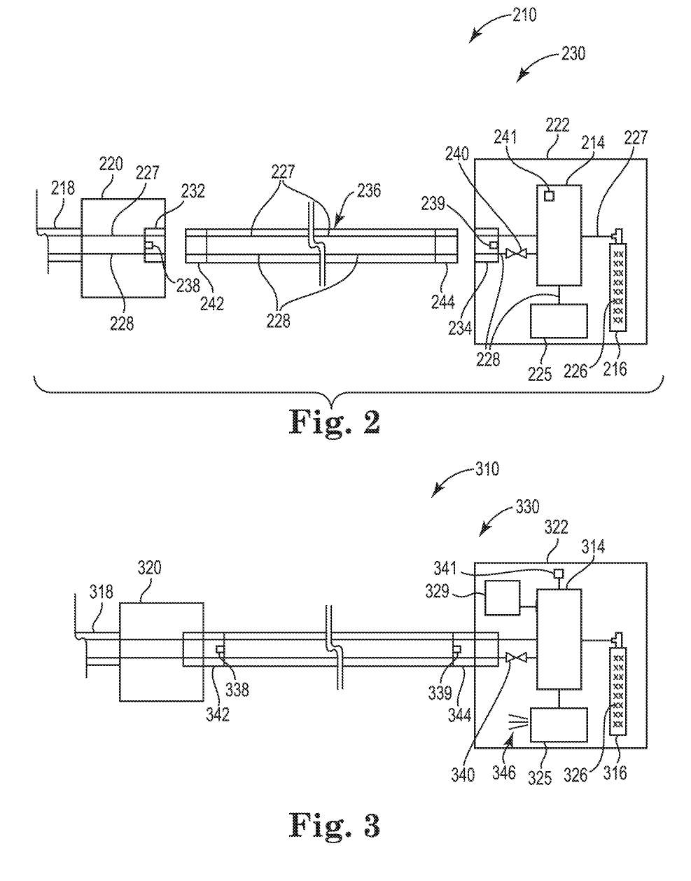

FIG. 2 is a simplified schematic side view of another embodiment of the catheter system 210 including one embodiment of a vacuum pump activation assembly 230. In the embodiment illustrated in FIG. 2, the catheter system 210 includes the control system 214, the fluid source 216, the balloon catheter 218, the handle assembly 220, the control console 222, the vacuum pump 225, a fluid injection line 227, a fluid exhaust line 228 and a vacuum pump activation assembly 230. Additionally, in FIG. 2, the vacuum pump activation assembly 230 is in a disassembled state. As used herein, “disassembled state” means that one or more of the structures and/or components of the vacuum pump activation assembly 230 and/or the catheter system 210, i.e., the handle assembly and/or control console 222, are detached, disengaged and/or separate from one or more other structures and/or components. In other words, when the vacuum pump activation assembly 230 is in the disassembled state, as shown in FIG. 2, the vacuum pump 225 is not activated and/or is not actively removing, evacuating and/or retrieving some or substantially all of the cryogenic fluid 226 from within the balloon catheter 218.

In FIG. 2, the handle assembly 220 includes a handle connector 232. In certain embodiments, the handle connector 232 can be integrated and/or positioned within and/or on the handle assembly 220. The design of the handle connector 232 can vary. In this embodiment, the handle connector 232 is configured to allow the handle assembly 220 to connect and/or couple, mechanically or otherwise, with the control console 222. Alternatively, the handle connector 232 can be configured to connect and/or couple the handle assembly 220 with any other suitable structure within the catheter system 210. Additionally, and/or alternatively, the handle connector 232 can allow the handle assembly 220 to connect and/or couple with the control console 222 and/or other structures via any suitable manner or method.

Additionally, in the embodiment illustrated in FIG. 2, the control console 222 includes a console connector 234. In certain embodiments, the console connector 234 can be integrated and/or positioned within and/or on the control console 222. The design of the console connector 234 can vary. In this embodiment, the console connector 234 is configured to allow the control console 222 to connect and/or couple, mechanically or otherwise, with the handle assembly 220. Alternatively, the console connector 234 can be configured to connect and/or couple the control console 222 with any other suitable structure within the catheter system 210. Additionally, and/or alternatively, the console connector 234 can allow the control console 222 to connect and/or couple with the handle assembly 220 and/or other structures via any suitable manner or method.

In certain embodiments, the fluid injection line 227 functions as a conduit through which the cryogenic fluid 226 is delivered from the fluid source 216 to the balloon catheter 218 during the cryoablation procedure. The design of fluid injection line 227 can vary. In the embodiment illustrated in FIG. 2, a portion of the fluid injection line 227 is shown to extend from the fluid source 216 to the balloon catheter 218. In alternative embodiments, the fluid injection line 227 can be connected to and/or extend through other structures and/or components of the catheter system 210.

In various embodiments, the fluid exhaust line 228 functions as a conduit through which cryogenic fluid 226 within the balloon catheter 218 can be vented, removed, evacuated and/or retrieved as exhaust (not shown) from the balloon catheter 218. The design of the fluid exhaust line 228 can vary. In the embodiment illustrated in FIG. 2, a portion of the fluid exhaust line 228 is shown to extend from the vacuum pump 225 to the balloon catheter 218. In alternative embodiments, the fluid exhaust line 228 can be connected to and/or extend through other structures and/or components of the catheter system 210.

The vacuum pump activation assembly 230 is configured to activate the vacuum pump 225. Specifically, in certain embodiments, once the vacuum pump activation assembly 230 determines that the handle assembly 220 is connected, mechanically or otherwise, with the control console 222, the vacuum pump activation assembly 230 can activate the vacuum pump 225. The vacuum pump activation assembly 230 can activate the vacuum pump 225 via any suitable manner and/or method. Additionally, in various embodiments, the vacuum pump activation assembly 230 can be configured to connect, mechanically or otherwise, the handle assembly 220 to the control console 222.

The design of the vacuum pump activation assembly 230 can vary depending on the design requirements of the catheter system 210. In the embodiment illustrated in FIG. 2, the vacuum pump activation assembly 230 can include one or more of a coupler 236, a first connection sensor 238, a second connection sensor 239, a control valve 240 and a controller 241. It is understood that the vacuum pump activation assembly 230 can include fewer or additional components than those specifically illustrated and described herein.

The coupler 236 couples and/or connects, mechanically or otherwise, the handle assembly 220 to the control console 222. The design of the coupler 236 can vary. In the embodiment illustrated in FIG. 2, the coupler 236 can include a portion of the fluid injection line 227, a portion of the fluid exhaust line 228, a first coupler connector 242 and a second coupler connector 244. It is understood that the coupler 236 can include fewer or additional components than those specifically illustrated and described herein.

In various embodiments, the portion of the fluid injection line 227 included within the coupler 236 can function as a conduit through which the cryogenic fluid 226 is delivered from the control console 222 to the handle assembly 220 during the cryoablation procedure. Further, the portion of the fluid exhaust line 228 included within the coupler 236 can function as a conduit through which cryogenic fluid 226 within the balloon catheter 218 can be vented, removed, evacuated and/or retrieved as exhaust from the handle assembly 220 to the control console 222.

In some embodiments, the first coupler connector 242 and/or second coupler connector 244 can be configured to allow the control console 222 to be connected and/or coupled, mechanically or otherwise, with the handle assembly 220, and vice versa. The design of the first coupler connector 242 and second coupler connecter 244 can vary. It is recognized that the terms “first coupler connector 242” and “second coupler connector 244” can be used interchangeably.

In certain embodiments, the first coupler connector 242 and/or second coupler connector 244 can be integrated and/or positioned within and/or on the coupler 236. More specifically, in one embodiment, the first coupler connector 242 and/or second coupler connector 244 can be configured to connect to the handle connector 232. In another embodiment, the first coupler connector 242 and/or second coupler connector 244 can be configured to connect to the console connector 234. Alternatively, the first coupler connector 242 and/or second coupler connector 244 can be configured to connect the coupler 236 with any other suitable structure within the catheter system 210. Additionally, and/or alternatively, the first coupler connector 242 and/or second coupler connector 244 can connect the coupler 236 to the handle assembly 220, the control console 222 and/or other structures of the catheter system 210 via any suitable manner and/or method.

In various embodiments, the first connection sensor 238 and/or the second connection sensor 239 can transmit or send electronic and/or other signals, e.g. sensor output, to the controller 241. In other words, the first connection sensor 238 can generate first sensor output and the second connection sensor 239 can generate second sensor output. For ease of reference, the sensor output generated by the first connection sensor 238 and/or the second connection sensor 239, i.e., first sensor output, second sensor output, etc., is referred to herein as “sensor output.” The design of the first connection sensor 238 and/or the second connection sensor 239 can vary. It is recognized that the terms “first connection sensor 238” and “second connection sensor 239” can be used interchangeably. In other words, either connection sensor 238, 239, can be the first connection sensor 238 or the second connection sensor 239. In some embodiments, the first connection sensor 238 and/or second connection sensor 239 can transmit or send sensor output to the controller 241 when the handle connector 232 and the first coupler connector 242 are connected. In other embodiments, the first connection sensor 238 and/or second connection sensor 239 can transmit or send sensor output to the controller 241 when the console connector 234 and the second coupler connector 244 are connected. In yet other embodiments, the first connection sensor 238 and/or second connection sensor 239 can transmit or send sensor output to the controller 241 when both the handle connector 232 and the first coupler connector 242 are connected, and the console connector 234 and the second coupler connector 244 are connected. While FIG. 2 shows two connection sensors 238, 239, it is understood that the catheter system 210 can include a plurality of different connection sensors 238, 239, i.e., first connection sensor, second connection sensor, third connection sensor, etc.

In certain embodiments, the first connection sensor 238 and/or second connection sensor 239 can directly sense when the handle connector 232 and the first coupler connector 242 are connected. In other embodiments, the first connection sensor 238 and/or second connection sensor 239 can directly sense when the console connector 234 and the second coupler connector 244 are connected. In still other embodiments, the first connection sensor 238 and/or second connection sensor 239 can directly sense when both the handle connector 232 and the first coupler connector 242 are connected, and the console connector 234 and the second coupler connector 244 are connected. As used herein, the term “directly” sense means that the first connection sensor 238 and/or second connection sensor 239 are in direct contact with the handle connector 232, the console connector, the first coupler connector 242 and/or the second coupler connector 244.

For example, in one embodiment, the first connection sensor 238 can include a first switch that can be selectively moved by the handle connector 232 and/or first coupler connector 242 when the handle connector 232 and/or first coupler connector 242 are connected. In another embodiment, the second connection sensor 239 can also include a second switch that can be selectively moved by the console connector 234 and/or the second coupler connector 244 when the console connector 234 and/or second coupler connector 244 are connected. In alternative embodiments, the first connection sensor 242 can include a first button that can be selectively depressed by the handle connector 232 and/or first coupler connector 242 when the handle connector 232 and/or first coupler connector 242 are connected. In other alternative embodiments, the second connection sensor 239 can also include a second button that can be selectively depressed by the console connector 234 and/or second coupler connector 244 when the console connector 234 and/or second coupler connector 244 are connected. Additionally, and/or alternatively, the first connection sensor 238 and/or second connection sensor 239 can include any other suitable design that effectively enables the first connection sensor 238 and/or second connection sensor 239 to directly sense when one or more of the handle connector 232, console connector 234, the first coupler connector 242 and/or the second coupler connector 244 are connected, and to transmit or send sensor output to the controller 241. The first connection sensor 238 and/or second connection sensor 239 can send the sensor output to the controller 241 via any suitable manner and/or method, such as a transmission line (not shown), as one non-exclusive example.

In non-exclusive alternative embodiments, the first connection sensor 238 and/or second connection sensor 239 can indirectly sense when the handle connector 232 is connected to the first coupler connector 242 and/or the console connector 234 is connected to the second coupler connector 244. In yet other alternative embodiments, the first connection sensor 238 and/or second connection sensor 239 can indirectly sense when both the handle connector 232 and the first coupler connector 242 are connected, and the console connector 234 and the second coupler connector 244 are connected. As used herein, the term “indirectly” sense means that the first connection sensor 238 and/or second connection sensor 239 are not in direct contact with the handle connector 232, the console connector 234, the first coupler connector 242 and/or the second coupler connector 244. As one non-exclusive example, the first connection sensor 238 may be located away from the handle connector 232 and/or first coupler connector 242, such as in the control console 222. In such embodiments, the first connection sensor 238 and/or second connection sensor 239 can sense that the handle connector 232 and the first coupler connector 242 are connected and/or the console connector 234 and the second coupler connector 244 are connected due to a change in fluid pressure. Alternatively, the first connection sensor 238 and/or second connection sensor 239 can indirectly sense when the handle connector 232 and the first coupler connector 242 and/or the console connector 234 and the second coupler connector 244 are connected via any suitable manner or method.

In the embodiment illustrated in FIG. 2, the first connection sensor 238 is located within handle connector 232. The first connection sensor 238 can be positioned at any location within the handle connector 232. Further, the second connection sensor 239 is located within the console connector 234. The second connection sensor 239 can be positioned at any location within the console connector 234. Alternatively, the first connection sensor 238 and/or second connection sensor 239 can be positioned at a location away from the handle connector 232 and/or console connector 234, i.e., at any other suitable location within the catheter system 210.

The control valve 240 can control the activation of the vacuum pump 225. In other words, the control valve 240 can control the venting, removal, evacuate and/or retrieve of the cryogenic fluid 226 from the balloon catheter 218. The control valve 240 can include any suitable type of valve. The vacuum pump activation assembly 230 can be configured to open and/or close the control valve 240. When the control valve 240 is open, the vacuum pump 625 is activated such that the vacuum pump 225 is pulling a vacuum and actively venting, removing, evacuating and/or retrieving some or all of the cryogenic fluid 228 from within the balloon catheter 218. Specifically, the vacuum pump activation assembly 230 can open the control valve 240 once the vacuum pump activation assembly 230 has determined that the handle assembly 220 is connected, mechanically or otherwise, with the control console 222. Contrarily, when the control valve 240 is closed, the vacuum pump 225 is not activated, i.e., the cryogenic fluid 228 is not being actively vented, removed, evacuated and/or retrieved from within the balloon catheter 218. The vacuum pump activation assembly 230 can open and/or close the control valve 240 via any suitable manner and/or method.

In the embodiment illustrated in FIG. 2, the control valve 240 is positioned on the fluid exhaust line 228. Further, the control valve 240 is located and/or positioned within the control console 222. The control valve 240 can be located and/or positioned on the fluid exhaust line 228 at any suitable location within control console 222.

The controller 241 is configured to receive and process the sensor output transmitted or sent from the first connection sensor 238 and/or second connection sensor 239. The design of the controller 241 can vary. In various embodiments, based at least in part on the sensor output, the controller 241 can process and/or determine whether the handle connector 232 and the first coupler connector 242 are connected and/or the console connector 234 and the second coupler connector 244 are connected. In some embodiments, the sensor output directly sensed and transmitted by the first connection sensor 238 and/or the second connection sensor 239 can then be processed by the controller 241 to open the control valve 240 to allow the vacuum pump 225 to actively vent, remove, evacuate and/or retrieve some or all of the cryogenic fluid 226 from within the balloon catheter 218, i.e., activate the vacuum pump 225. In other embodiments, the sensor output indirectly sensed and transmitted by the first connection sensor 238 and/or the second connection sensor 239 can also then be processed by the controller 241. In such other embodiments, the controller 241 can process the sensor output indirectly sensed to open the control valve 241, i.e., activate the vacuum pump 225. The controller 241 can process the sensor output, whether directly or indirectly sensed, via any suitable method. Further, in the embodiment illustrated in FIG. 2, the controller 241 can be included as part of the control system 214. In other embodiments, the controller 241 can be separate from the control system 214.

In alternative embodiments, connecting and/or engaging the handle connector 232 and/or first coupler connector 242 and the console connector 234 and/or the second coupler connector 244 can cause the control valve 240 to mechanically open. In addition, disconnecting and/or disengaging the handle connector 232 and/or first coupler connector 242 and the console connector 234 and/or the second coupler connector 244 can cause the control valve 240 to mechanically close. In such alternative embodiments, the control valve 240 can mechanically open and/or close via any suitable manner and/or method.

FIG. 3 is a simplified schematic side view of still another embodiment of the catheter system 310 including another embodiment of the vacuum activation assembly 330. In the embodiment illustrated in FIG. 3, the catheter system 310 includes the control system 314, the fluid source 316, the balloon catheter 318, the handle assembly 320, the control console 322, the graphical display 324, the vacuum pump 325 and the vacuum pump activation assembly 330. In the embodiment illustrated in FIG. 3, the vacuum pump activation assembly 330 is in an assembled state. As used herein, “assembled state” means that the structures and/or components of the vacuum pump activation assembly 330, the handle assembly 320 and the control console 522, are attached, engaged and/or connected. In other words, when the vacuum pump activation assembly 330 is in the assembled state, as shown in FIG. 3, the vacuum pump 325 can be activated to actively and/or intermittently remove, evacuate and/or retrieve some or substantially all of the cryogenic fluid 326 from within the balloon catheter 318.

In this embodiment, the controller 341 is separate from the control system 314, and integrated and/or included as part of the control console 322. In various embodiments, based at least in part on the sensor output, the controller 341 can process and/or determine whether the handle assembly 320 and the control console 322 have been connected, mechanically or otherwise, via the vacuum pump activation assembly 330. In other words, the first connection sensor 338 and/or second connection sensor 339 can directly and/or indirectly sense when the vacuum pump activation assembly 330 is in the assembled state, and transmit or send the sensor output to the controller 341. Once the controller 341 has processed and/or determined that the handle assembly 320 and the control console 322 are connected via the vacuum pump activation assembly 330, as shown in FIG. 3, the controller 341 can open the control valve 340 to allow the vacuum pump 325 to actively vent, remove, evacuate and/or retrieve some or all of the cryogenic fluid 326 from within the balloon catheter 318, i.e., activate the vacuum pump 325 based at least in part on the sensor output received from one or both of the first connection sensor 338 and/or the second connection sensor 339. In this embodiment, the first connection sensor 338 is located within the first coupler connector 342 and the second connection sensor 339 is located within the second coupler connector 344.

In certain embodiments, by opening the control valve 340, the vacuum pump 325 pulls a vacuum. For example, during cryoablation procedures, the vacuum pump 325 can remove, evacuate and/or retrieve some of the cryogenic fluid 326 from the balloon catheter 318 to assist in maintaining an appropriate balloon pressure within the balloon catheter 318. At or near completion of the cryoablation procedure, the vacuum pump 325 can further remove, evacuate and/or retrieve most or substantially all of the cryogenic fluid 326 from within the balloon catheter 318 to allow the balloon catheter 318 to be more safely removed from the patient 12 (illustrated in FIG. 1). The controller 341 can open the control valve 340 to activate the vacuum pump 325 to remove, evacuate and/or retrieve some or substantially all of the cryogenic fluid 326 within the balloon catheter 318 via any suitable manner or method.

In various embodiments, once the vacuum pump 325 has been activated, the vacuum pump activation assembly 330 can also operate to assist in detecting breaches within the balloon catheter 318. More particularly, the first connection sensor 338 and/or second connection sensor 339 can sense whether an error or breach exists and/or is present within the balloon catheter 318 in an area between an inner cryoballoon (not shown) and an outer cryoballoon (not shown) and can transmit or send sensor output to the controller 341. The first connection sensor 338 and/or second connection sensor 339 can sense the existence and/or presence of the error and/or breach via any suitable manner and/or method. In certain embodiments, upon receipt of the sensor output, the controller 341 can process and subsequently cause the graphical display 324 to identify and/or notify the operator whether or not the cryoablation procedure is ready to be performed. In one embodiment, the vacuum pump activation assembly 330 can include a breach sensor (not shown) that detects breaches within the balloon catheter 318 and generates sensor output. In this embodiment, upon receipt of the sensor output, the controller 341 can close the control valve 340 and cause the graphical display 324 to alert and/or notify the operator.

In the embodiment illustrated in FIG. 3, a cryogenic fluid exhaust 346 is vented and/or emptied from the vacuum pump 325 within the control console 322. In other embodiments, the cryogenic fluid exhaust 346 can be vented and/or emptied from other suitable structures within the catheter system 310. In alternative embodiments, the cryogenic fluid exhaust 346 can be vented and/or emptied outside of the control console 322. Additionally, and/or in the alternative, the cryogenic fluid exhaust 346 can be vented and/or emptied via any suitable manner and/or method.

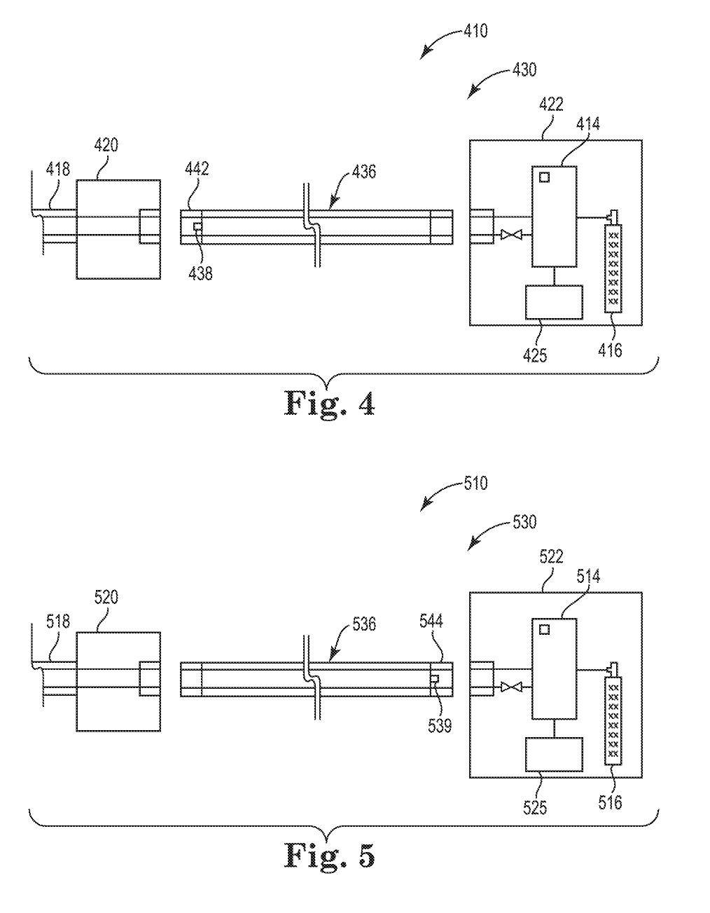

FIG. 4 is a simplified side view of yet another embodiment of the catheter system 410 including still another embodiment of the vacuum pump activation assembly 430. In the embodiment illustrated in FIG. 4, the catheter system 410 includes the control system 414, the fluid source 416, the balloon catheter 418, the handle assembly 420, the control console 422, the vacuum pump 425 and the vacuum pump activation assembly 430. However, in the embodiment illustrated in FIG. 4, the vacuum pump activation assembly 430 only includes the first connection sensor 438. In this embodiment, the first connection sensor 438 is located and/or positioned within the first coupler connector 442. Alternatively, the first connection sensor 438 can be located and/or positioned at any location within the coupler 436. Additionally, and/or alternatively, the first connection sensor 438 can be integrated and/or included within any structure and/or component of the catheter system 410.

FIG. 5 is a simplified side view of even another embodiment of the catheter system 510 including yet another embodiment of the vacuum pump activation assembly 530. In the embodiment illustrated in FIG. 5, the catheter system 510 includes the control system 514, the fluid source 516, the balloon catheter 518, the handle assembly 520, the control console 522, the vacuum pump 525 and the vacuum pump activation assembly 530. However, in this embodiment, the vacuum pump activation assembly 530 only includes the second connection sensor 539. In this embodiment, the second connection sensor 539 is located and/or positioned within the second coupler connector 544. Alternatively, the second connection sensor 539 can be located and/or positioned at any location within the coupler 536. Additionally, and/or alternatively, the second connection sensor 539 can be integrated and/or included within any suitable structure and/or component of the catheter system 510.

It is appreciated that the embodiments of the vacuum pump activation assembly described in detail herein enable the realization of one or more certain advantages during the cryoablation procedure. With the various designs illustrated and described herein, the vacuum pump activation assembly can decrease the likelihood of operator error by automatically activating the vacuum pump when the vacuum pump activation assembly is in the connected state.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A catheter system comprising:
a balloon catheter configured to receive a cryogenic fluid and including a handle assembly that includes a handle connector;
a control console that includes a console connector; a vacuum pump; and
a vacuum pump activation system comprising:
a coupler that includes a first coupler connector and a second coupler connector, the first coupler connector being configured to connect to the handle connector, the second coupler connector being configured to connect to the console connector;
a first connection sensor that senses when at least one of (i) the first coupler connector and the handle connector are connected, and (ii) the second coupler connector and the console connector are connected, the first connection sensor generating a first sensor output;
a control valve that is positioned on the fluid exhaust line; and
a controller that receives the first sensor output, the controller selectively opening the control valve to activate the vacuum pump based at least partially upon the first sensor output.

2. The catheter system of claim 1, wherein the first connection sensor is positioned within the handle connector.

3. The catheter system of claim 1, wherein the first connection sensor is positioned within the first coupler connector.

4. The catheter system of claim 1, wherein the first connection sensor is positioned within the console connector.

5. The catheter system of claim 1, wherein the first connection sensor is positioned within the second coupler connector.

6. The catheter system of claim 1, wherein the control valve is located within the control console.

7. The catheter system of claim 1, further comprising a second connection sensor that senses when at least one of (i) the first coupler connector and the handle connector are connected, and (ii) the second coupler connector and the console connector are connected, the second connection sensor generating a second sensor output.

8. The catheter system of claim 7, wherein the controller receives the second sensor output, the controller selectively opening the control valve to activate the vacuum pump based at least partially upon the second sensor output.

9. A vacuum pump activation system for a catheter system, the catheter system including (i) a handle assembly that includes a handle connector, (ii) a balloon catheter, the balloon catheter being configured to receive a cryogenic fluid, (iii) a control console that includes a console connector, and (iv) a vacuum pump, the vacuum pump activation system comprising:
a coupler that includes a first coupler connector and a second coupler connector, the first coupler connector being configured to connect to the handle connector, the second coupler connector being configured to connect to the console connector;
a first connection sensor that senses when the first coupler connector and the handle connector are connected, the first connection sensor generating a first sensor output;
a second connection sensor that senses when the second coupler connector and the console connector are connected, the second connection sensor generating a second sensor output;
a control valve that is positioned on the fluid exhaust line; and
a controller that receives the first sensor output and second sensor output, the controller selectively opening the control valve to activate the vacuum pump based at least partially upon the first sensor output and the second sensor output.

10. The vacuum pump activation system of claim 9, wherein the first connection sensor is positioned within the handle connector.

11. The vacuum pump activation system of claim 9, wherein the first connection sensor is positioned within the first coupler connector.

12. The vacuum pump activation system of claim 9, wherein the second connection sensor is positioned within the console connector.

13. The vacuum pump activation system of claim 9, wherein the second connection sensor is positioned within the second coupler connector.

14. The vacuum pump activation system of claim 9 wherein the control valve is located within the control console.

15. A method for activating a vacuum pump, the method comprising:
connecting a first coupler connector to a handle connector and a second coupler connector to a console connector;
generating first sensor output with a first connection sensor;
sending the first sensor output generated by the first connection sensor to a controller; and
opening with the controller a control valve based at least partially upon the first sensor output.

16. The method of claim 15, wherein sending the first sensor output includes the first connection sensor being positioned within the handle connector.

17. The method of claim 15, wherein sending the first sensor output includes the first connection sensor being positioned within the first coupler connector.

18. The method of claim 15, wherein sending the first sensor output includes the first connection sensor being positioned within the console connector.

19. The method of claim 15, wherein sending the first sensor output includes the first connection sensor being positioned within the second coupler connector.

20. The method of claim 15, further comprising:
generating second sensor output with a second connection sensor; and
opening a control valve based at least partially upon the second sensor output.
